(19) United States
(12) Patent Application Publication
(10) Pub. No.: US 2019/0275196 A1
(43) Pub. Date: Sep. 12, 2019

(54) Hemostatic Powders with Self-Assembling Peptide Hydrogels

(71) Applicants: Eun Seok GIL, Waltham, MA (US); Eoton ALEKSI, West Roxbury, MA (US); 3-D Matrix, Ltd., Tokyo (JP)

(72) Inventors: Eun Seok Gil, Lexington, MA (US); Elton Aleksi, West Roxbury, MA (US); Marc Rioult, Walpole, MA (US)

(21) Appl. No.: 16/304,928

(22) PCT Filed: Jun. 1, 2017
(86) PCT No.: PCT/US2017/035431

§ 371 Date: Nov. 27, 2018

Related U.S. Application Data
(60) Provisional application No. 62/344,181, filed on Jun. 1, 2016.

(51) Int. Cl. A61L 24/04; A61L 24/10; A61L 24/00; A61B 17/00

(52) U.S. Cl. CPC A61L 24/043; A61L 24/108; A61L 24/0042; A61L 24/0031; A61L 2400/04; A61B 2017/00495; A61L 2300/418; A61L 2400/06; A61B 17/00491

(57) ABSTRACT

Hemostatic powders are synergistically used in conjunction with self-assembling peptide hydrogels to promote hemostasis at a target site. Related methods, kits, and devices for hemostasis are disclosed.

### FIELD OF THE TECHNOLOGY

One or more aspects relate to hemostatic powders used in conjunction with self-assembling peptide hydrogels for various medical, research, and industrial applications.

### BACKGROUND

Hemostasis generally relates to the prevention of blood loss from vessels and organs of the body of a subject. The process plays an important role in stopping or otherwise controlling blood flow during surgery, medical treatment, and wound healing. While hemostasis is a natural biological process involving coagulation, various chemical, mechanical, and physical agents may be implemented to achieve or promote hemostasis.

### SUMMARY

In accordance with one or more aspects, a kit for hemostasis may comprise a solution comprising a self-assembling peptide comprising between about 7 amino acids and 32 amino acids in an effective amount and in an effective concentration for use in forming a hydrogel under physiological conditions to promote hemostasis, and a hemostatic powder miscible in the solution.

In some aspects, the self-assembling peptide may be selected from the group consisting of RADA16, IEIK13, and KLD12. The hemostatic powder may comprise microspheres and/or micro-fibrils. The hemostatic powder may comprise a bio-absorbable material. The hemostatic powder may comprise collagen, gelatin, chitosan, polysaccharide, starch, hyaluronic acid, silk fibroin, or oxidized regenerated cellulose. In some aspects, the hemostatic powder may comprise a synthetic biomaterial. The synthetic biomaterial may be selected from the group consisting of: Poly(lactide-co-glycolide) (PLGA), (PLGA)-poly(ethylene glycol)-block-copolymer, and (PLGA-b-PEG).

In some aspects, the kit may further comprise a syringe system for mixing the solution and the hemostatic powder. The kit may further comprise instructions for administering the solution and the hemostatic powder to a target site. The instructions may provide direction to mix the solution and the hemostatic powder in a ratio of about 0.1 to 20 mL solution per 1 g hemostatic powder by weight. In some non-limiting aspects, the instructions may provide direction to mix the solution and the hemostatic powder in a ratio of about 0.5 to 7 mL solution per 1 g hemostatic powder by weight. The instructions may provide direction to apply a mixture of the solution and the hemostatic powder to the target site in excess, and then to cover the target site with gauze. The instructions may still provide further direction to apply tactile pressure to the gauze.

In some aspects, the kit may further comprise at least one of: a luer-lock syringe, a delivery nozzle, a bottle, a spreader, a container, and gauze. An inner diameter of the delivery nozzle may be about 0.5 mm to about 10 mm, and a length of the nozzle may be from about 0.5 cm to about 30 cm. The nozzle may be flexible.

In accordance with one or more aspects, a macroscopic scaffold may consist essentially of a hemostatic powder and a plurality of self-assembling peptides, each of the self-assembling peptides comprising between about 7 amino acids and about 32 amino acids in an effective amount to promote hemostasis at a target area.

In some embodiments, the kit and/or macroscopic scaffold provide hemostasis to a target area having a bleeding score of 2 or more on the WHO Bleeding Scale. In some embodiments, the kit and/or macroscopic scaffold may provide hemostasis to a target area in 2 minutes or less. Specifically, the kit and/or macroscopic scaffold may reduce a bleeding score of a target area to 0 on the WHO Bleeding Scale in 2 minutes or less. In some embodiments, the kit and/or macroscopic scaffold may provide hemostasis to a target area having an initial bleeding score of 3 or 4 on the WHO Bleeding Scale in 2 minutes or less, for example, upon applying a mixture of the self-assembling peptide and hemostatic powders disclosed herein.

Still other aspects, embodiments, and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. The accompanying drawings are included to provide illustration and a further understanding of the various aspects and embodiments, and are incorporated in and constitute a part of this specification. The drawings, together with the remainder of the specification, serve to explain principles and operations of the described and claimed aspects and embodiments.

### BRIEF DESCRIPTION OF THE DRAWINGS

The accompanying drawings are not intended to be drawn to scale. For purposes of clarity, not every component may be labeled. In the drawings:

FIG. 1 includes six images of a process for using a hemostatic powder with a self-assembling peptide hydrogel, according to one embodiment;

FIG. 2 is a visualization of gel formation in conjunction with hemostatic powders, according to another embodiment;

FIG. 3 is an alternate visualization of gel formation in conjunction with hemostatic powders, according to another embodiment;

FIG. 4 is a graph of storage/loss modulus of gelatin powder and saline;

FIG. 5 is a graph of storage/loss modulus of a self-assembling peptide hydrogel;

FIG. 6 is a graph of storage/loss modulus of mixtures of gelatin powder and a self-assembling peptide hydrogel, according to certain embodiments;

FIG. 7 is an alternate graph of storage modulus of various mixtures described herein, according to certain embodiments;

FIG. 8 is a visualization of gel formation in conjunction with hemostatic powders, according to another embodiment;

FIG. 9 is an alternate visualization of gel formation in conjunction with hemostatic powders, according to another embodiment;

FIG. 10 includes three images of wound defect sites treated with a hemostatic powder and/or a self-assembling peptide, according to embodiments described herein;

FIG. 11 is a graph of the degree of bleeding (bleeding score) over time of samples treated with a hemostatic powder and saline, thrombin, or a self-assembling peptide hydrogel, according to certain embodiments described herein; and

FIG. 12 is a graph of hemostatic success (%) over time achieved in the samples treated with a hemostatic powder and saline, thrombin, or a self-assembling peptide hydrogel, according to certain embodiments described herein.

### DETAILED DESCRIPTION

In accordance with one or more embodiments, self-assembling peptide hydrogels may be used as a scaffold for hemostasis. PuraMatrix® peptide hydrogel (hereinafter “PuraMatrix®”), commercially available from 3-D Matrix Co., Ltd., for example, is a synthetic, 16-amino acid polypeptide with a repeating sequence of arginine, alanine, and aspartic acid, or RADARADARADARADA (RADA16). PuraMatrix® is known to self-assemble to form a hydrogel under physiological conditions and can be used for various biomedical applications. In accordance with various embodiments described herein, PuraMatrix® may be used for hemostatasis. PuraStat® is a synthetic peptide hydrogel also commercially available from 3-D Matrix Co., Ltd. Other relevant non-limiting synthetic peptide sequences may be represented by self-assembling peptides having the repeating sequence of lysine, leucine, and aspartic acid (Lys-Leu-Asp (KLD)), and such peptide sequences are represented by (KLD)p, wherein p=2-50, such as KLD12. Still other relevant non-limiting synthetic peptide sequences may be represented by self-assembling peptides having the repeating sequence of isoleucine, glutamic acid, isoleucine and lysine (Ile-Glu-Ile-Lys (IEIK)), and such peptide sequences are represented by (IEIK)p, wherein p=2-50, such as IEIK13. Other embodiments may involve still other self-assembling peptides. In some non-limiting embodiments, peptide hydrogels such as those disclosed in International Patent Application Publication No. WO2015/138514 titled “SELF-ASSEMBLING PEPTIDE COMPOSITIONS” and assigned to 3-D Matrix, Ltd., which is hereby incorporated herein by reference in its entirety for all purposes, may be implemented.

Embodiments disclosed herein may comprise certain peptide compositions (and particularly certain compositions of self-assembling peptide agents), and technologies relating thereto. In some embodiments, such compositions may be or comprise solutions. In some embodiments, such compositions may be or comprise gels. In some embodiments, such compositions may be or comprise solid (e.g., dried/lyophilized) peptides. For example, particular peptide compositions (i.e., peptide compositions having specific concentration, ionic strength, pH, viscosity and/or other characteristics) have useful and/or surprising attributes (e.g., gelation or self-assembly kinetics [e.g., rate of gelation and/or rate and reversibility of peptide self-assembly], stiffness [e.g., as assessed via storage modulus], and/or other mechanical properties).

In some embodiments, peptides included in provided compositions are self-assembling peptides. In some embodiments, peptides included in provided compositions are amphiphilic peptides. In some embodiments, peptides included in provided compositions have an amino acid sequence characterized by at least one stretch (e.g., of at least 4, 5, 6, 7, 8, 9, 10, 11, 12, 13, 14, 15, 16, 17, 18, 19, 20 etc amino acids) of alternating hydrophilic and hydrophobic amino acids. In accordance with one or more embodiments, peptide compositions may include an amphiphilic polypeptide having about 6 to about 200 amino acid residues. In some embodiments, a peptide may have a length within the range of about 6 to about 20 amino acids and an amino acid sequence of alternating hydrophobic amino acid and hydrophilic amino acids.

In some embodiments, peptides included in provided compositions have an amino acid sequence that includes one or more repeats of Arg-Ala-Asp-Ala (RAD A). In some embodiments, peptides included in provided compositions have an amino acid sequence that comprises or consists of repeated units of the sequence Lys-Leu-Asp-Leu (KLDL). In some embodiments, peptides included in provided compositions have an amino acid sequence that comprises or consists of repeated units of the sequence Ile-Glu-Ile-Lys (IEIK). In some embodiments, the peptides may be IEIK13, KLD12, or RADA16. In some embodiments, compositions of these peptides may have enhanced properties relative to appropriate reference compositions that have different (e.g., lower) pH level, and/or ionic strength.

In some embodiments, increased ionic strength may beneficially impact stiffness and/or gelation kinetics to peptide compositions rendering them suitable for a broader range of applications. In some embodiments, increased ionic strength may be physiological ionic strength, which may occur when peptide compositions are placed into the body. In some embodiments, an ionic strength of a peptide composition may be about 0.0001 M to about 1.5 M. In some embodiments, an ionic strength of a peptide composition may be adjusted by mixing common salts, for example, NaCl, KCl, MgCl₂, CaCl₂, CaSO₄, DPBS (Dulbecco's Phosphate-Buffered Saline, 10×). In some embodiments, ionic strengths of peptide compositions may be adjusted by mixing common salts, wherein one or more common salts are composed of one or more salt forming cations and one or more salt forming anions, wherein the salt forming cations are selected from the group consisting of ammonium, calcium, iron, magnesium, potassium, pyridinium, quaternary ammonium, and sodium, wherein the salt forming anions are selected from the group consisting of acetate, carbonate, chloride, citrate, cyanide, floride, nitrate, nitrite, and phosphate.

In accordance with one or more aspects, properties of certain peptide compositions, including but not limited to IEIK13, KLD12, and RADA16, may be enhanced by maintaining their pH level at about 3.5 or less and, at the same time, their salt concentrations at less than their critical ionic strength levels (i.e. no precipitation). In some embodiments, a peptide composition may have a pH within the range of about 2.5 to about 4.0, or within the range of about 3.0 to about 4.0. In some embodiments, provided compositions have a pH at or above about 2.5, 2.6, 2.7, 2.8, 2.9, 3.0, about 3.1, about 3.2, about 3.3, about 3.4, about 3.5 or higher. In some embodiments, provided compositions have a pH at or below about 4.3, about 4.2, about 4.1, about 4.0, about 3.9, about 3.7, about 3.6, about 3.5, about 3.4, or lower. In some embodiments, pH of a peptide composition can be achieved with a solution selected from the group consisting of sodium hydroxide or, potassium hydroxide, calcium hydroxide, sodium carbonate, sodium acetate, sodium sulfide, DMEM (Dulbecco's modified Eagle's medium), and PBS (Phosphate-Buffered Saline).

In some embodiments, a peptide composition may be solution, gel, or any combination thereof. In some embodiments, peptide concentration in a peptide composition is at least 0.05%, at least 0.25%, at least 0.5%, at least 0.75%, at least 1.0% or more. In some embodiments, peptide concentration in a peptide composition is less than 5%, less than 4.5%, less than 4%, less than 3.5%, less than 3%, or less. In some embodiments, peptide concentration in a peptide composition is within a range between about 0.5% and about 3%. In some embodiments, peptide concentration in a peptide composition is within a range between about 0.5% and about 2.5%. In some embodiments, peptide concentration in a peptide composition is within a range between about 1% and about 3%. In some embodiments, peptide concentration in a peptide composition is within a range between about 1% and about 2.5%. In some embodiments, peptide concentration in a peptide composition is about 0.5%, about 1%, about 1.5%, about 2%, about 2.5%, about 3%, or more. In some particular embodiments, where the peptide is RADA16, peptide concentration in peptide compositions is within a range of about 0.05% to about 10%.

In some embodiments, a peptide composition may have a viscosity with the range of about 1 to about 10000 Pa-S. In some embodiments, a peptide composition may have a storage modulus with the range of about 50 to about 2500 Pa.

The term “peptide” as used herein refers to a polypeptide that is typically relatively short, for example, having a length of less than about 100 amino acids, less than about 50 amino acids, less than 20 amino acids, or less than 10 amino acids.

The term “polypeptide” as used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide.

In some embodiments, the term “polypeptide” may be appended to a name of a reference polypeptide, activity, or structure. In such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known. In some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class. In some embodiments, a member of a polypeptide class or family shows significant sequence homology or identity, shares a common sequence motif, and/or shares a common activity with all polypeptides within the class.

For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90%> or even 95%, 96%, 97%, 98%, or 99%. Such a conserved region usually encompasses at least 3-4 and often up to 20 or more amino acids. In some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

The term “self-assembling” is used herein in reference to certain polypeptides that, under appropriate conditions, can spontaneously self-associate into structures. For example, such that solutions (e.g., aqueous solutions) containing them develop gel character. In some embodiments, interactions between and among individual self-assembling polypeptides within a composition are reversible, such that the composition may reversibly transition between a gel state and a solution state. In some embodiments, self-assembly (and/or dis-assembly) is responsive to one or more environmental triggers (e.g., change in one or more of pH, temperature, ionic strength, osmolarity, osmolality, applied pressure, applied shear stress, etc). In some embodiments, compositions of self-assembling polypeptides are characterized by detectable beta-sheet structure when the polypeptides are in an assembled state.

In accordance with one or more embodiments, self-assembling peptide hydrogels may be used with a hemostatic powder as a scaffold for hemostasis. In accordance with one or more aspects, the hemostatic properties of various hemostatic powders may be enhanced by using them in conjunction with self-assembling peptide hydrogels. In accordance with one or more further aspects, the hemostatic properties of self-assembling peptide hydrogels may be enhanced by using them in conjunction with various hemostatic powders. Various embodiments described herein are therefore directed to the synergy exhibited by concurrent use of hemostatic powders and self-assembling peptide hydrogels for hemostasis.

In some embodiments disclosed herein, self-assembling peptide hydrogels used with hemostatic powders may provide hemostasis to a target area experiencing heavy bleeding, upon applying a mixture of self-assembling peptide hydrogel solution and hemostatic powder. For instance, the mixture may be applied to the wound with a gauze, while applying tactile pressure to the top of the gauze over the wound.

Hemostasis is the first stage of wound healing. As disclosed herein “hemostasis” is used to reference a reduction in bleeding. For example, hemostasis may refer to a reduction in bleeding of an open wound. In some embodiments, hemostasis is defined as a complete stop in bleeding. In some embodiments, hemostasis is defined as a significant stop in bleeding. Generally, hemostasis refers to a visually significant reduction in bleeding of an open wound.

In accordance with certain embodiments, the self-assembling peptide hydrogels used with hemostatic powders, as disclosed herein, may be used to stop heavy bleeding. For instance, embodiments disclosed herein may stop bleeding of a scale of 2 or higher on the World Health Organization (WHO) Bleeding Scale. The WHO Bleeding Scale is a clinical investigator-assessed five-point scale with 0=No bleeding, 1=Petechiae, 2=Mild blood loss, 3=Gross blood loss, and 4=Debilitating blood loss. Embodiments disclosed herein may be used to treat wounds classified as producing mild blood loss (2 on the WHO scale), gross blood loss (3 on the WHO scale), or debilitating blood loss (4 on the WHO scale).

In accordance with certain embodiments, hemostasis is achieved when bleeding is a 1 or lower on the WHO scale. For instance, hemostasis may be achieved when bleeding is visually determined to be a 1, 0.5, or 0 on the WHO bleeding scale. For instance, in some embodiments disclosed herein, self-assembling peptide hydrogels used with hemostatic powders may reduce bleeding of a target area to a bleeding score of 0.5 or less on the WHO Bleeding Scale, upon applying the mixture and tactile pressure to a top of a gauze at the target area. Self-assembling peptide hydrogels used with hemostatic powders may reduce bleeding of a target area to a bleeding score of 0 on the WHO Bleeding Scale, for example after 2 minutes of applying the mixture and tactile pressure to the target area.

In accordance with one or more non-limiting embodiments, the self-assembling peptide hydrogel may be IEIK13, KLD12, or RADA16. The self-assembling peptide may comprise between about 7 amino acids and 32 amino acids in an effective amount and in an effective concentration for use in forming a hydrogel under physiological conditions to promote hemostasis. In some specific embodiments, the self-assembling peptide may comprise between about 12 to about 16 amino acids that alternate between a hydrophobic amino acid and a hydrophilic amino acid. The peptide hydrogel may gel upon contact with blood to stop and/or control bleeding via mechanical blocking of a bleeding site. Upon gelation, a resulting peptide hydrogel may be substantially transparent so as to allow unobstructed viewing of a target area. The peptide hydrogels may generally be characterized as non-biogenic, biocompatible, and resorbable. The self-assembling peptide hydrogel may be present in solution at varying concentrations. For example, in some non-limiting embodiments, a 2.5% peptide hydrogel solution may be used. In other, non-limiting embodiments, a 1.3% peptide hydrogel solution may be used. In at least some embodiments, the solution may be substantially free of cells and/or drugs. In other embodiments, the solution may include one or more therapeutic agents to promote hemostasis. As described further herein, the solution may be formulated, such as to impact its stiffness and/or gelation kinetics, or to provide a suitable environment for an intended application.

Generally, self-assembling peptide hydrogels alone may be used to treat bleeding of a scale of 1 or less on the WHO Bleeding Scale. When directly applied to a wound or treatment site, a self-assembling peptide hydrogel, substantially free of agents, and used without mixing with a hemostatic powder, the peptide hydrogel may not be effective at achieving hemostasis of a heavy bleeding wound site. For instance, a self-assembling peptide hydrogel, with nothing more, may not stop heaving bleeding of a scale of 3 or 4 on the WHO Bleeding Scale. Accordingly, while self-assembling peptide hydrogels may be used as a scaffold for hemostasis, and may be capable of achieving hemostasis of certain wounds, the peptide hydrogels, generally, may not achieve hemostasis of wounds classified as having gross or debilitating blood loss (3 or 4 on the WHO scale). Embodiments disclosed herein, which combine self-assembling peptide hydrogels and a hemostatic powder in a miscible mixture, may synergistically achieve hemostasis of wounds having blood loss of a 2 or greater on the WHO Bleeding Scale.

In accordance with one or more embodiments, a target pH level and/or tonicity level for the solution may be selected at least in part based on the type of cell or tissue involved in an intended application. For example, a pH level of the peptide hydrogel may be adjusted to a level of up to about 3.0, for example, up to a level of about 3.4 or 3.5, for improved cell viability by providing a more gentle, less harsh environment. With respect to tonicity, the tonicity of a peptide hydrogel solution may be adjusted so as to closely match the plasma osmolality of a target cell type and/or target species. For example, the tonicity of the peptide hydrogel solution may be adjusted based on the plasma osmolality of any given cell type. Tonicity levels may range depending on the type of species and/or the type of cell or tissue involved. In some non-limiting embodiments, a target tonicity may range from about 260 to about 360 mOsm/L.

Generally, a number of therapeutic sites may be treated as described herein. A therapeutic site may refer to a site of injury. Therapeutic sites may be exterior or interior sites. Exterior therapeutic sites include superficial and/or exterior bleeding sites or open wounds experiencing blood loss of a scale of 2 or higher on the WHO Bleeding Scale. Exterior therapeutic sites may include sites of trauma or amputation. Interior sites may include surgical incisions made on exposed tissues experiencing a blood loss of a scale of 2 or higher on the WHO bleeding scale. Interior sites may include surgical incisions for the purpose of surgical treatment, or internal bleeding sites that have been at least partially exposed for treatment. In some embodiments, interior sites include therapeutic sites treated by endoscopic and/or laparoscopic procedures.

In accordance with one or more embodiments, the hemostatic powder may generally be miscible in the solution. The hemostatic powder may include microspheres and/or micro-fibrils.

In some embodiments, the hemostatic powder may be made of a bio-absorbable material. For example, the hemostatic powder may include collagen, gelatin, chitosan, polysaccharide, starch, hyaluronic acid, silk fibroin, or oxidized regenerated cellulose. In some embodiments, the hemostatic powder may be a synthetic biomaterial. For example, the hemostatic powder may include Poly(lactide-co-glycolide) (PLGA), (PLGA)-poly(ethylene glycol)-block-copolymer, or (PLGA-b-PEG). In accordance with one or more embodiments, the hemostatic powder may be Surgiflo® hemostatic powder commercially available from Ethicon, Floseal® hemostatic powder commercially available from Baxter, Gelform® hemostatic powder commercially available from Pfizer, Arista® hemostatic powder commercially available from Medafor, or Helitene® hemostatic powder commercially available from Integra.

Hemostatic powders are generally capable of stopping heavy blood flow from large wounds. For instance, when applied with tactile pressure on a gauze over a wound site, hemostatic powders may stop hemorrhage from large arteries and veins within several minutes of application. Hemostatic powders disclosed herein, when applied without a self-assembling peptide hydrogel, may achieve hemostasis from a heavily bleeding wound (3 or 4 on the WHO scale) in about 5 to about 8 minutes. When used with a self-assembling peptide hydrogel, as described herein, hemostatic powders and hydrogels may achieve hemostasis from a similar heavily bleeding wound in about 5 minutes or less. Specifically, embodiments disclosed herein may provide hemostasis to a target area having a bleeding score of 3 or 4 on the WHO Bleeding Scale in 2 minutes or less. Generally, hemostatic powders and self-assembling peptides may be applied to the target area in a mixture, for example, with tactile pressure applied to the top of a gauze over the wound.

As noted above, the peptide hydrogel and the hemostatic powder may be used in conjunction in accordance with various embodiments. This combination may beneficially impart relatively fast and easy delivery of the peptide hydrogel solution to a target location, such as a wound area or a surgical site, in comparison to alternative approaches such as those involving sole application. This combination may also beneficially impart assistance with respect to the application of hand or finger pressure, which can be applied on the top of applied powder to temporarily hold bleeding flow which, in turn, may achieve stable gelation of the self-assembling peptide hydrogel near the bleeding wound surface without hindrance by the bleeding flow. The combination may also beneficially provide a reservoir space in the voids among powder particles which may contain peptide solution so as to allow for the release of reserved peptide solution onto the wound when it is squeezed by a hand or finger. Peptide hydrogel may at the same time be retained in the reservoir space to cover a target area. The viscosity of the peptide hydrogel solution may also beneficially impart a sticky property which may cause the hemostatic powder to more stably stay in position on a target area.

In accordance with one or more embodiments, the peptide solution and the hemostatic powder may be used in a ratio of about 0.1 to 20 mL solution per 1 g hemostatic powder by weight. For instance, the peptide solution and hemostatic powder may be used in a ratio of about 0.1 mL, 0.2 mL, 0.5 mL, 1.0 mL, 2.5 mL, 5 mL, 7.5 mL, 10 mL, 12.5 mL, 15 mL, 17.5 mL, 18 mL, 19 mL, or 20 mL of solution per 1 g of hemostatic powder by weight. In accordance with one or more specific non-limiting embodiments, the peptide solution and the hemostatic powder may be used in a ratio of about 0.5 to 7 mL solution per 1 g hemostatic powder by weight. The peptide hydrogel solution and/or hemostatic powder may be provided in the kit in a volume exceeding the volume requirement for the therapeutic site.

In accordance with one or more embodiments, a hemostatic powder and a peptide solution may be combined and provided together as a single device. The device may include a solution and a hemostatic powder miscible in the solution. The solution may comprise a self-assembling peptide. The self-assembling peptide may comprise between about 7 amino acids and 32 amino acids in an effective amount and in an effective concentration for use in forming a hydrogel under physiological conditions to promote hemostasis. The device may be prepackaged for use at a target area. The packaging may include instructions for administering the device to a target area for hemostasis. For example, the instructions may provide direction to apply the mixture of the solution and the hemostatic powder to a target site in excess, and then to cover the target site with gauze. The instructions may further involve direction to apply tactile pressure to a top of the applied device at the target area, or to the gauze covering it.

In accordance with one or more other embodiments, a kit for hemostasis may alternatively be provided. The kit may include both a hemostatic powder and a peptide hydrogel solution. The two components may be packaged together in the kit. Instructions for use may also be provided. The instructions may provide guidance for how to mix the peptide hydrogel solution to the hemostatic powder prior to or during use in connection with a target area at a predetermined ratio. The kit may include one or more further components to facilitate the combination of the hemostatic powder and the peptide hydrogel solution prior to or during use. For example, such components may include devices for combining and delivering self-assembling peptide hydrogel and powders. In accordance with one or more embodiments, the devices may include a syringe, such as one with a male or female luer-lock, which contains self-assembling peptide solution and another syringe, such as another with a male or female luer-lock, which contains hemostatic powders. The two syringes may then be connected with their luer-locks for mixing the two materials by pushing the respective plungers back and force several times until the consistency is substantially uniform. In accordance with one or more embodiments, the devices may include a nozzle to deliver the mixture of peptide solution and hemostatic powders to a target area. In accordance with one or more non-limiting embodiments, an inner diameter of the nozzle may be from 0.5 mm to 10 mm and a length of the nozzle may be from 0.5 cm to 30 cm. In accordance with one or more embodiments, the nozzle may be flexible to be curved to apply the material to a variety of positions. In accordance with one or more embodiments, the kit may include a gauze or other protective covering, which can be used to cover the mixture applied at a target area, such as during application of finger or hand pressure. The kit may include instructions for administering a mixture of hemostatic powder and peptide hydrogel to a target area for hemostasis. The instructions may further involve direction to apply tactile pressure at the target area.

In still other embodiments, a hemostatic powder and a peptide hydrogel solution may be packaged and provided separately from each other. Each may be packaged as a separate product and then combined prior to or during use. One or both separately packaged components may include instructions for administering the hemostatic powder and peptide hydrogel to a target area for hemostasis. The instructions may further involve direction to apply tactile pressure to a top of the applied mixture at the target area. One or both separately packaged components may also optionally include additional components such as those described above to facilitate the concurrent usage, including but not limited to the one or more syringes and nozzles. In accordance with one or more non-limiting embodiments, a macroscopic scaffold may consist essentially of a hemostatic powder and a plurality of self-assembling peptides, each of the self-assembling peptides comprising between about 7 amino acids and about 32 amino acids in an effective amount to promote hemostasis at a target area.

The function and advantages of these and other embodiments will be more fully understood from the following non-limiting examples. The examples are intended to be illustrative in nature and are not to be considered as limiting the scope of the embodiments discussed herein.

### EXAMPLES

### Example 1

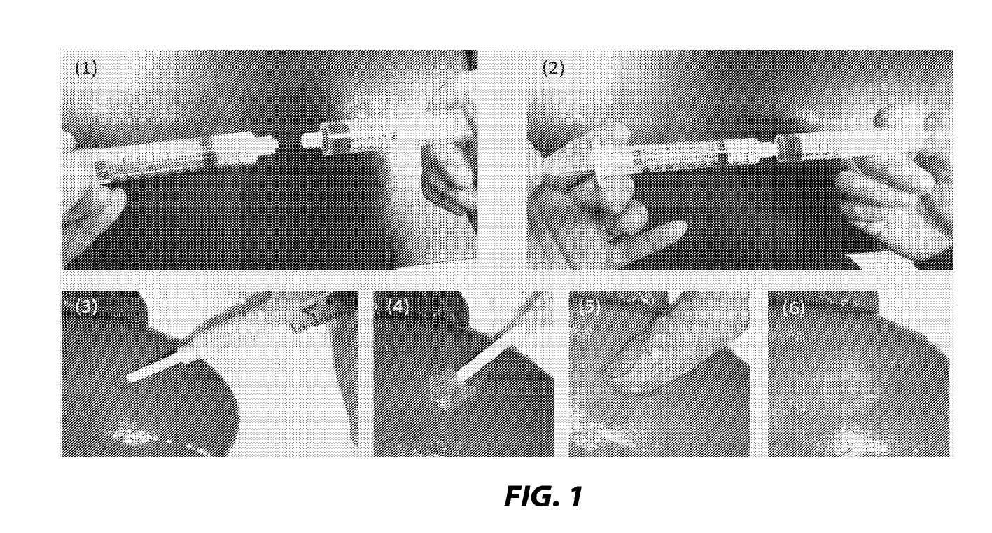

This example illustrates the use of certain hemostatic powders with certain self-assembling peptide hydrogels with reference to FIG. 1 as discussed herein. In (1), absorbable gelatin powder (Surgiflo®, Ethicon) in a syringe with a female luer-lock and self-assembling peptide (PuraMatrix®) in another syringe with a male luer-lock are provided. In (2), the two syringes are connected and mixed by pushing their plungers back and forth, for example, six times. In (3), bleeding is observed at a target site, blood is removed from the target site, and the mixture of gelatin powder and PuraMatrix® is applied to the target site. In (4), an excess amount of the mixture is provided at the target site. In (5), pressure is applied over the mixture by finger or hand until hemostasis is achieved. A gauze can be used to cover the material and the wound before applying pressure. In (6), hemostasis is achieved.

### Example 2

The capability of a peptide hydrogel to gelate when used in conjunction with hemostatic powders was demonstrated. A Congo Red assay was performed to determine gel formation of peptide solutions in a saline buffer solution (pH 7.4) when used with hemostatic powders.

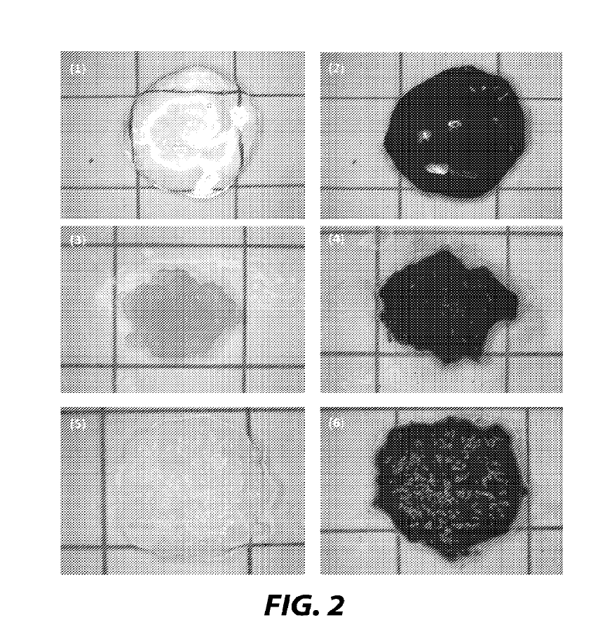

Pure self-assembling peptide solution (PuraMatrix®), and peptide solution/hemostatic powders (Surgiflo®, Ethicon) mixtures were plated on a glass slide. After 30 seconds, 1% Congo Red solution in saline buffer solution (pH 7.4) was added around and on top of the gel aliquots and then the excess Congo Red solution was wiped off prior to examination. Visualization of gel formation determined the success or failure of gelation. As shown in FIG. 2, the self-assembling peptide solutions gelled even when mixed with hemostatic powder to a similar extent as observed in pure peptide solution. In (1) and (2), self-assembling peptide solution (PuraMatrix®) before and after Congo red assay, respectively is shown. In (3) and (4), PuraMatrix® mixed with absorbable gelatin powder (Surgiflo®, Ethicon) at a ratio of 2 to 1 (v/w) before and after Congo red assay, respectively is shown. In (5) and (6), PuraMatrix® mixed with absorbable gelatin powder (Surgiflo®, Ethicon) at a ratio of 5 to 1 (v/w) before and after Congo red assay, respectively is shown.

Accordingly, as shown in FIG. 2, RADA16 2.5% is capable of gelation when mixed with a hemostatic powder at a ratio of 2 to 1 and when mixed with a hemostatic powder at a ratio of 5 to 1. The gelated self-assembling peptide and hemostatic powder combination may be capable of promoting hemostasis on a bleeding wound.

### Example 3

The capability of a peptide hydrogel to gelate when used in conjunction with hemostatic powders was demonstrated. A Congo Red assay was performed to determine gel formation of peptide solutions in a saline buffer solution (pH 7.4) when used with hemostatic powders.

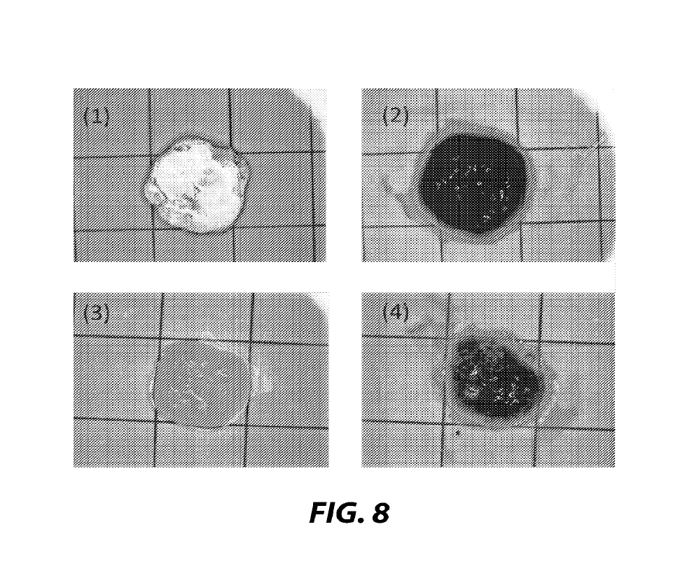

Pure self-assembling peptide solution (IEIK13 1.3% at pH3.0) and peptide solution/hemostatic powders (Surgiflo®, Ethicon) mixture were plated on a glass slide. After 30 seconds, 1% Congo Red solution in saline buffer solution (pH 4.7) was added around and on top of the gel aliquots and then the excess Congo Red solution was wiped off prior to examination. Visualization of gel formation determined the success or failure of gelation. As shown in FIG. 8, the self-assembling peptide solution gelled even when mixed with hemostatic powder to a similar extent as observed in pure peptide solution. In (1) and (2), self-assembling peptide solution (IEIK13 1.3% at pH 3.0) before and after Congo Red assay, respectively, is shown. In (3) and (4), IEIK13 1.3% at pH 3.0 mixed with absorbable gelatin powder, (Surgiflo®, Ethicon) at a ratio of 2 to 1 (v/w) before and after Congo Red assay, respectively, is shown. The interval of grids in FIG. 8 is 1 cm.

Accordingly, as shown in FIG. 8, IEIK13 1.3% (pH3.0) is capable of gelation when mixed with a hemostatic powder at a ratio of 2 to 1. It is expected that IEIK13 1.3% (pH 3.0) will be capable of gelation when mixed with a hemostatic powder at a ratio of 5 to 1, as observed with RADA16 2.5%. The gelated self-assembling peptide and hemostatic powder combination may be capable of promoting hemostasis on a bleeding wound.

### Example 4

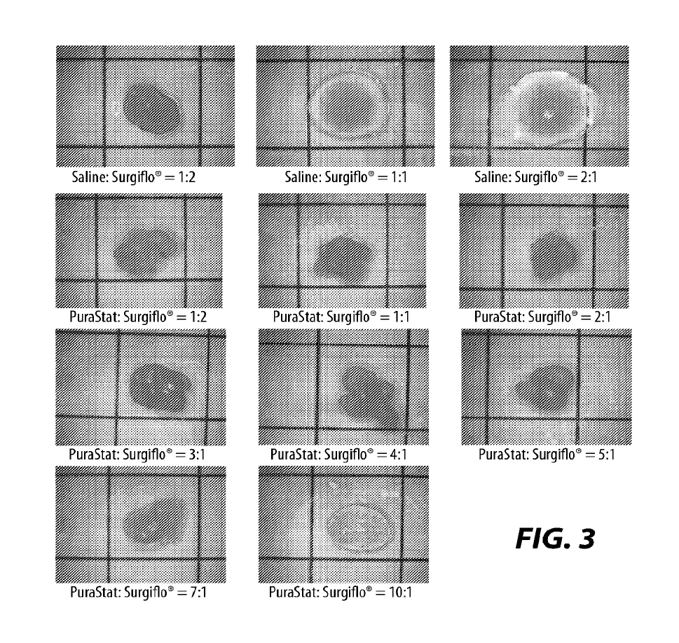

Homogenous mixing of self-assembling peptide solutions and gelatin powders at various mixing ratios in comparison to saline was demonstrated. Gelatin powder (Surgiflo®) was separately mixed with saline and RADA16 2.5% solution (PuraMatrix®) to determine their apparent miscibility. Gelatin powders were placed in a luer-lock syringe and saline or RADA16 2.5% solution (PuraMatrix®) was placed in another luer-lock syringe. The syringes were connected to mix the contents of the two syringes by moving the plungers back and forth, for example, six times until the consistency was even. The mixtures were plated on a glass slide. FIG. 3 presents images of gelatin powder (Surgiflo®) and saline mixtures (upside images) and gelatin powder and RADA16 2.5% (PuraMatrix®) mixtures at various mixing ratios. As shown in FIG. 3, RADA16 and gelatin powders were homogeneously mixed across various mixing ratios, while saline and gelatin powders were not well mixed when the content of gelatin powders was lower.

Accordingly, as shown in FIG. 3, RADA16 2.5% is capable of homogenous mixture when combined with a hemostatic powder. The homogeneously mixed and gelated self-assembling peptide and hemostatic powder combination may be capable of promoting hemostasis on a bleeding wound.

### Example 5

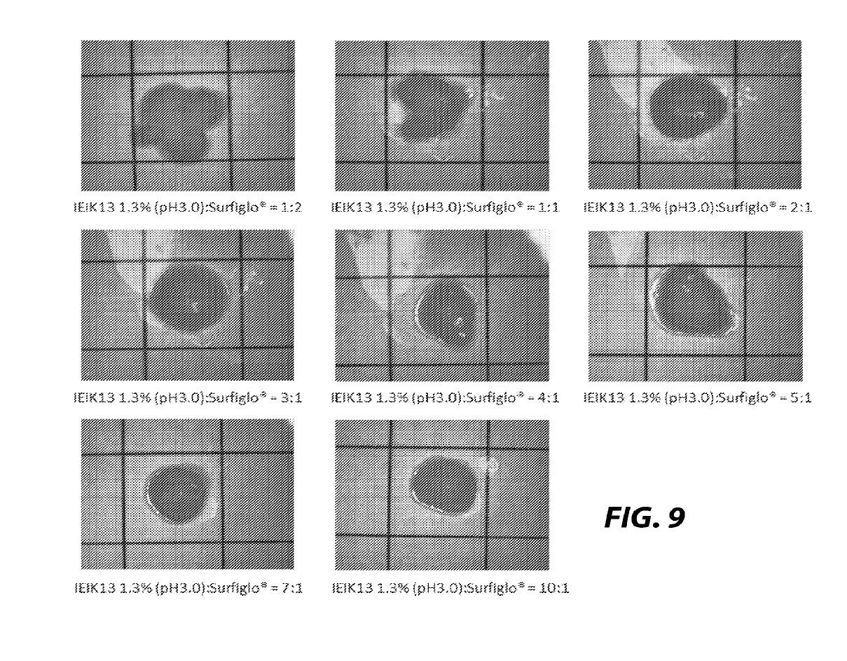

Homogenous mixing of self-assembling peptide solutions and gelatin powders at various mixing ratios in comparison to saline was demonstrated. Gelatin powder (Surgiflo®) was mixed with IEIK13 1.3% (pH 3.0) solution to determine its apparent miscibility. Gelatin powders were placed in a luer-lock syringe and IEIK13 1.3% (pH 3.0) was placed in another luor-lock syringe. The syringes were connected to mix contents of the two syringes by moving the plungers back and forth, for example, six times until the consistency was even. The mixtures were plated on a glass slide. FIG. 9 presents images of gelatin powder (Surgiflo®) and IEIK13 mixtures at various mixing ratios. As shown in FIG. 9, IEIK13 and gelatin powders were homogeneously mixed across various mixing ratios. Comparatively, and as shown in FIG. 3, saline and gelatin powders were not well mixed when the content of gelatin powders was lower.

Accordingly, as shown in FIG. 9, IEIK13 1.3% (pH3.0) is capable of homogenous mixture when combined with a hemostatic powder. The homogeneously mixed and gelated self-assembling peptide and hemostatic powder combination may be capable of promoting hemostasis on a bleeding wound.

### Example 6

The rheological properties of gelatin powders with saline, self-assembling peptide, and gelatin powder with self-assembling peptide were evaluated using a rheometer (DHR-1, TA Instruments) with 20 mm plates. The samples were placed on the rheometer plate and the moduli were measured at 25° C. with the plates placed at a measuring geometry gap of 1000 μm. Measurements were performed after 2 minutes of relaxation time at 25° C. Frequency sweep tests were performed at 1 rad/sec˜10 red/sec of oscillation stress with strain at 0.01.

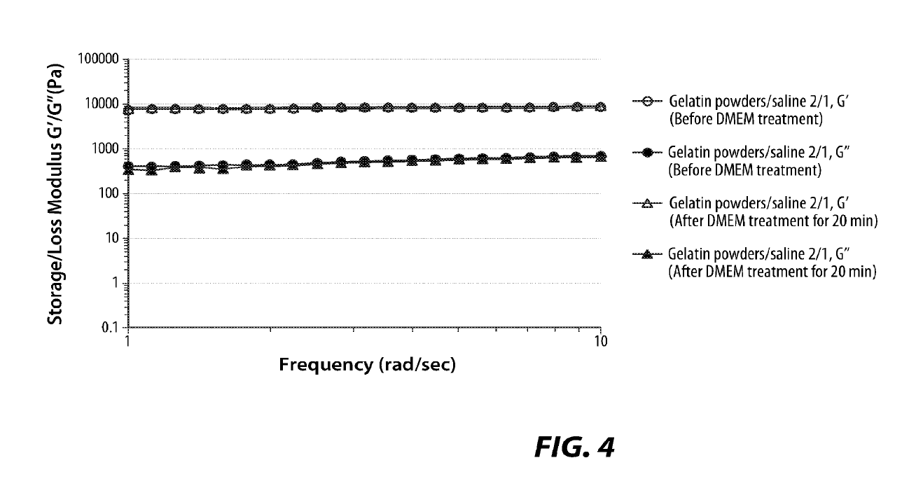

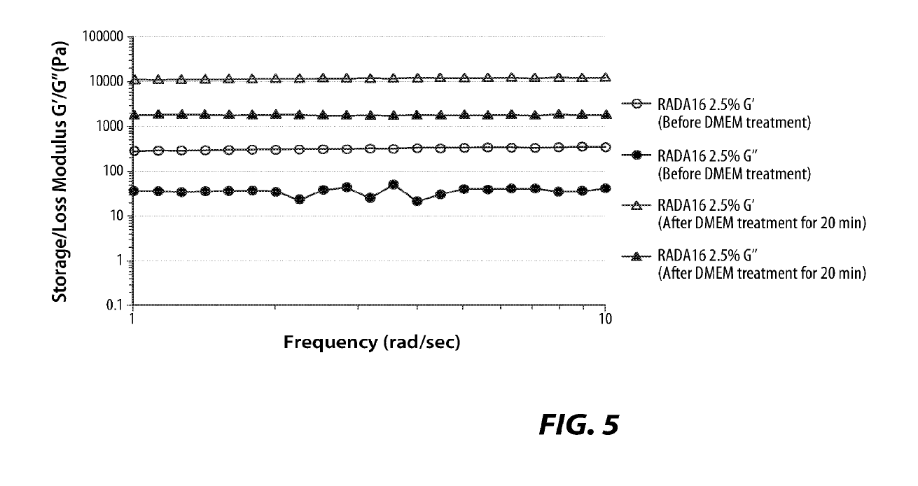

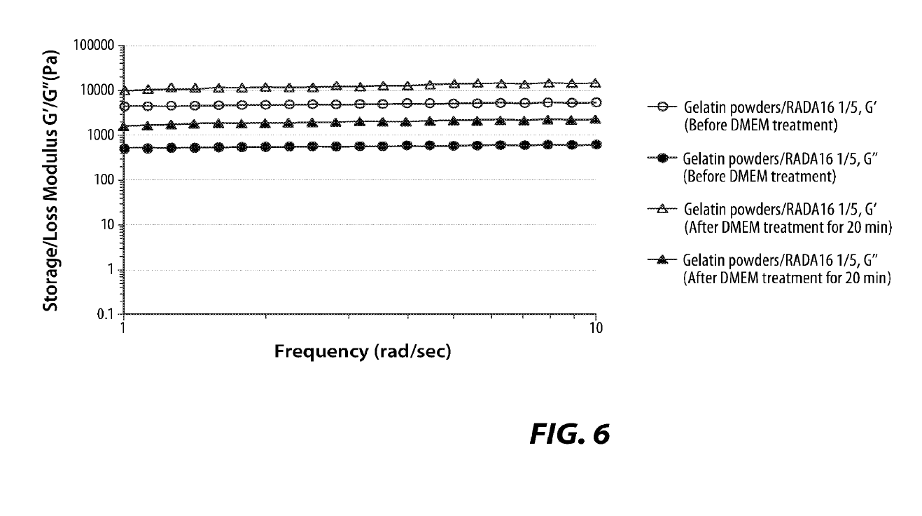

Pure RADA16 2.5% (PuraMatrix®), gelatin powder (Surgiflo®) mixed with saline at 2:1 w/v ratio, and gelatin powder mixed with RADA16 2.5% at various mixing ratios were all tested. These samples were treated with DMEM for 20 min after their frequency tests were performed without DMEM treatment. The storage and loss modulus plots of these samples before and after DMEM treatment are shown in FIGS. 4-6. FIG. 4 presents the rheology of gelatin powders mixed with saline at a ratio of 2:1 w/v before and after DMEM treatment. FIG. 5 presents the rheology of RADA16 2.5% solution before and after DMEM treatment. FIG. 6 presents the rheology of gelatin powders mixed with RADA16 2.5% at a ratio of 1:5 w/v before and after DMEM treatment.

As shown in FIGS. 4 and 5, RADA16 2.5% solution and gelatin powders with saline at a 2:1 w/v ratio as described in the instruction of Surgiflo® were tested as controls. As shown in FIG. 6, after gelatin powder mixed with RADA16 2.5% at a 1:5 ratio was tested, the moduli of gelatin powder mixed with saline did not change after DMEM treatment. However, the moduli of gelatin powders mixed with RADA16 2.5% increased after DMEM treatment as shown in pure RADA16 2.5%. Thus, even when mixed with gelatin powder, RADA16 formed a gel.

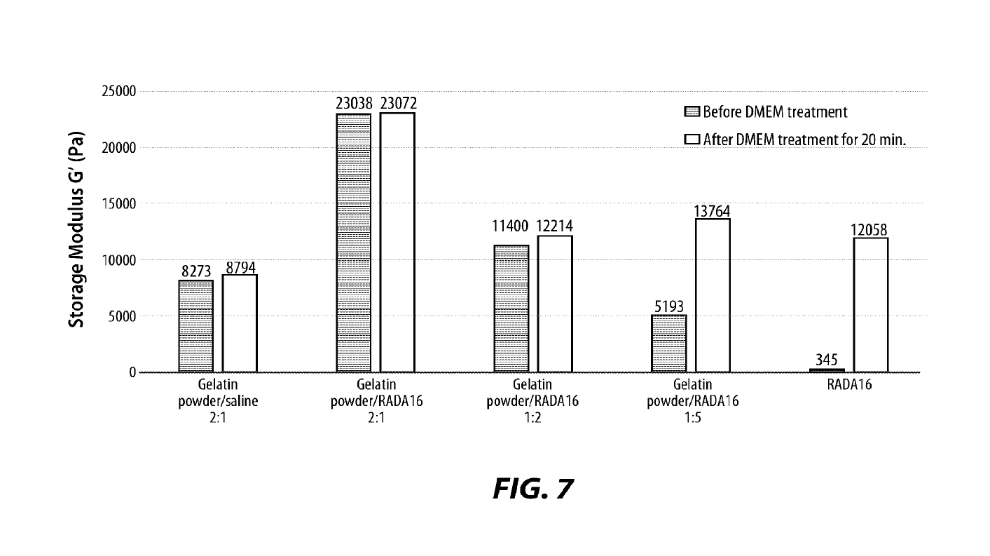

FIG. 7 presents rheology data showing the storage moduli of gelatin powders with self-assembling peptide at different ratios before and after DMEM treatment. Before gelation, the moduli of gelatin powders mixed with RADA16 2.5% increased with more gelatin powder. However, the moduli of gelatin powder mixed with RADA16 2.5% increased more predominantly with more RADA16 2.5% when they were treated with DMEM. Change in the moduli upon DMEM treatment was more significant with increased RADA 16 2.5% content. The moduli of gelatin powder with RADA16 2.5% at a ratio of 2:1 w/v was 2.8 times higher than that of gelatin powder with saline at a ratio of 2:1 w/v.

### Example 7

The following comparative example illustrates the enhanced hemostatic efficacy of a gelatin powder when utilized with a self-assembling peptide hydrogel. Specifically, the comparative example further illustrates the similarity in effectiveness between a gelatin powder with thrombin solution and a gelatin powder with self-assembling peptide solution.

A study was performed to evaluate the efficacy of hemostatic agents in an organ wounding model in swine. A midline laparotomy was performed on each animal model. The liver was exposed and isolated. Multiple bleeding defects were created using a punch biopsy across the three lobes of the liver. An 8 mm biopsy punch instrument was used to create a circular defect that was approximately 2-5 mm in depth. All liver sites resulted in acceptable bleeding scores (3-4 on the WHO Bleeding Scale) following biopsy punch and prior to test article application.

Test samples were prepared with gelatin powder (Surgiflo®, Ethicon). The gelatin powder was mixed with 2 mL or 4 mL of RADA16 2.5% surgical hemostatic agent (PuraStat®). Test samples were also prepared by mixing the gelatin powder with 2 mL of a thrombin solution. Thrombin is clinically used as a surgical hemostat. Generally, thrombin may be used in conjunction with other hemostatic agents, for example, absorbable sponges, collagen, cellulose, and fibrinogen. However, thrombin is an unfavorable agent because it may cross react with human coagulation factors (if foreign in origin) or it may transmit blood-borne pathogens and be limited in availability (if human in origin). Accordingly, there exists a need for a hemostatic solution that is safe for use in surgery and also widely available.

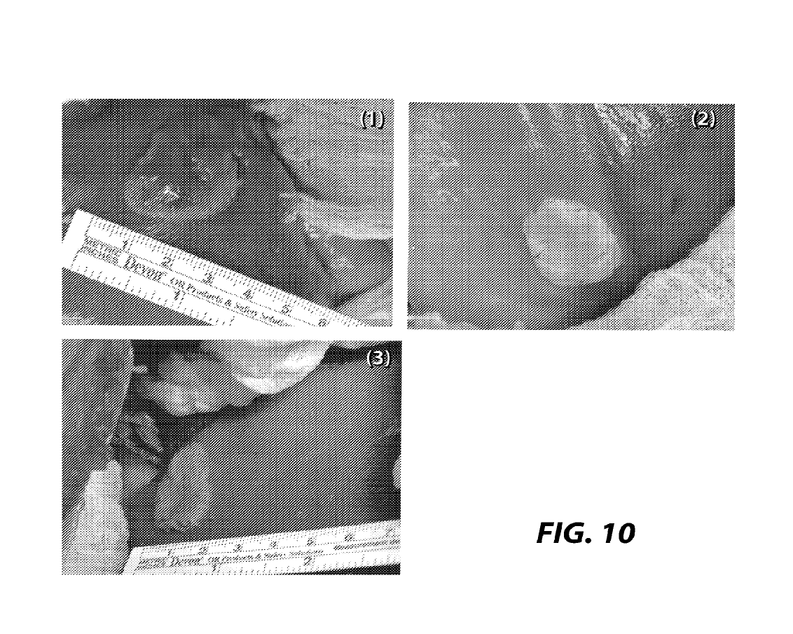

The hemostatic samples of the experiment were applied to each wound site on a saline dampened gauze. Control hemostatic samples were prepared mixing the gelatin powder with 2 mL of saline and applied to wound sites similarly to the test samples (on a saline dampened gauze). Test samples were applied in a volume sufficient to cover the entire defect site of each wound, as shown in FIG. 10. In (1) a Surgiflo® and saline control sample was applied to the liver biopsy defect. In (2) a Surgiflo® and thrombin test sample was applied to the liver biopsy defect. In (3) a Surgiflo® and PuraStat® test sample was applied to the liver biopsy defect.

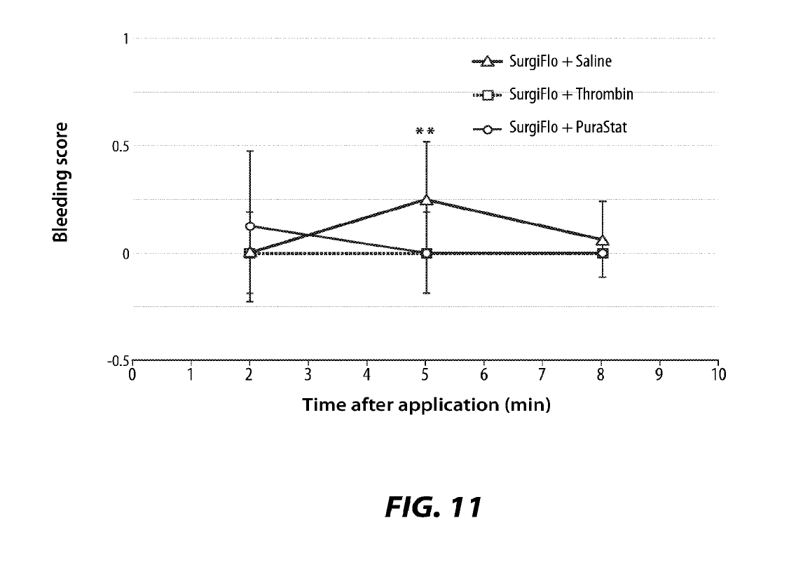

Each test sample was applied to the liver wound site on the saline dampened gauze with pressure for approximately 2 minutes. The liver lesions were scored for bleeding immediately following the two minute pressure application period, at 5 minutes after application, and at 8 minutes after application. The results are summarized in the graph of FIG. 11. No significant difference was found for initial bleeding score (time=0) between the different sites treated with test and control samples. Specifically, initial bleeding of all samples was determined to be a 3 or 4 on the WHO bleeding scale.

Bleeding was reduced in all test article preparation sites following the 2 minutes of article application with direct pressure. Test articles treated with Surgiflo®+thrombin and Surgiflo®+PuraStat® resulted in lower bleeding scores at 2 minutes and at 5 minutes after article application, as compared to the test articles of Surgiflo®+saline. No significant differences were found among the test samples at 2 minutes after application and at 8 minutes after application. No significant differences were found between Surgiflo®+thrombin and SurgiFlo®+PuraStat® at all time points tested. Surgiflo®+thrombin and Surgiflo®+PuraStat® exhibited no bleeding at 8 minutes after application, while Surgiflo®+saline showed 1 bleeding site at 8 minutes after application, among 8 sites treated. Notably, the Surgiflo®+PuraStat® hemostatic effect superiority over Surgiflo®+saline hemostatic effect is especially significant at 5 minutes after application (p<0.05). Specifically, after 5 minutes, the sites treated with Surgiflo®+saline exhibited an average bleeding of 0.25 on the WHO bleeding scale, while the sites treated with Surgiflo®+PuraStat® exhibited an average bleeding of 0 on the WHO bleeding scale.

The data show no significant superiority of Surgiflo®+thrombin over Surgiflo®+PuraStat® at all time points tested. Specifically, even though each of the sites treated with Surgiflo®+thrombin exhibited an average bleeding of 0 on the WHO bleeding scale at all time points tested, after 2 minutes, the sites treated with Surgiflo®+PuraStat® exhibited an average bleeding of only 0.13 on the WHO bleeding scale, and after 5 and 8 minutes the Surgiflo®+PuraStat® sites exhibited an average bleeding of 0 on the WHO bleeding scale.

The bleeding scores of Surgiflo®+saline are summarized in Table 1, Surgiflo® and thrombin in Table 2, and Surgiflo®+PuraStat® in Table 3.

TABLE 1
Bleeding Scores of Surgiflo ® + saline samples.

| | Bleeding score | | | |
|---|---|---|---|---|
| | Initial bleeding | | | |
| | score before | After application | | |
| Sample # | application | 2 min | 5 min | 8 min |
| 1 | 3 | 0 | 0.5 | 0 |
| 2 | 4 | 0 | 0 | 0 |
| 3 | 4 | 0 | 0.5 | 0.5 |
| 4 | 4 | 0 | 0.5 | 0 |
| 5 | 4 | 0 | 0 | 0 |
| 6 | 3 | 0 | 0.5 | 0 |
| 7 | 4 | 0 | 0 | 0 |
| 8 | 4 | 0 | 0 | 0 |
| (Mean, SD) | (3.75, 0.46) | (0, 0) | (0.25, 0.27) | (0.06, 0.17) |

TABLE 2
Bleeding Scores of Surgiflo ® + thrombin samples.

| | Bleeding score | | | |
|---|---|---|---|---|
| | Initial bleeding | | | |
| | score before | After application | | |
| Sample # | application | 2 min | 5 min | 8 min |
| 1 | 4 | 0 | 0 | 0 |
| 2 | 4 | 0 | 0 | 0 |
| 3 | 4 | 0 | 0 | 0 |
| 4 | 4 | 0 | 0 | 0 |
| 5 | 4 | 0 | 0 | 0 |
| 6 | 3 | 0 | 0 | 0 |
| 7 | 4 | 0 | 0 | 0 |
| 8 | 3 | 0 | 0 | 0 |
| (Mean, SD) | (3.75, 0.46) | (0, 0) | (0, 0) | (0, 0) |

TABLE 3
Bleeding Scores of Surgiflo ® + PuraStat ® samples.

| | Bleeding score | | | |
|---|---|---|---|---|
| | Initial bleeding | | | |
| | score before | After application | | |
| Sample # | application | 2 min | 5 min | 8 min |
| 1 | 4 | 0 | 0 | 0 |
| 2 | 4 | 1 | 0 | 0 |
| 3 | 4 | 0 | 0 | 0 |
| 4 | 4 | 0 | 0 | 0 |
| 5 | 4 | 0 | 0 | 0 |
| 6 | 3 | 0 | 0 | 0 |
| 7 | 4 | 0 | 0 | 0 |
| 8 | 4 | 0 | 0 | 0 |
| (Mean, SD) | (3.88, 0.35) | (0.13, 0.35) | (0, 0) | (0, 0) |

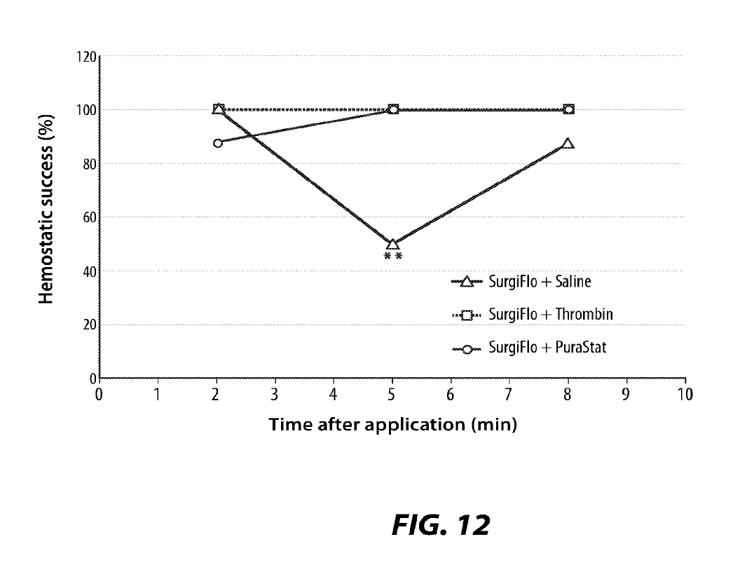

The graph of FIG. 12 shows hemostatic success (%) after application. The bleeding score of all Surgiflo®+thrombin and Surgiflo®+PuraStat® samples after 8 minutes was 0 (100% hemostatic success). Surgiflo®+PuraStat® samples showed a higher hemostatic success at 5 minutes and 8 minutes after application (each 100%), as compared to Surgiflo®+saline (50% and 87.5%, respectively). Specifically, at 5 and 8 minutes after application, 8 of the 8 defect sites treated with Surgiflo® and PuraStat® had achieved hemostasis, as compared to 4 of 8 defect sites that achieved hemostasis with Surgiflo®+saline at 5 minutes, and 7 of 8 that achieved hemostasis with Surgiflo®+saline at 5 minutes. The Z score test for two population proportions demonstrates the significant superiority of Surgiflo®+PuraStat® over Surgiflo®+saline. There was no significant superiority of Surgiflo®+thrombin over Surgiflo®+PuraStat®, as each of the samples exhibited 100% hemostatic success at 5 minutes and 8 minutes after application.

Similar results are expected with an IEIK13 1.3% (pH 3.0) self-assembling peptide hydrogel, due to the similar gelation mechanics of IEIK13 1.3% (pH 3.0) and RADA16 2.5%, as shown above in Examples 2-5.

Accordingly, a self-assembling peptide hydrogel can be utilized with a gelatin powder. The self-assembling powder can enhance the hemostatic efficacy of a gelatin powder to a similar degree as unfavorable and non-widely available thrombin. Furthermore, the self-assembling peptide can enhance the hemostatic efficacy of the gelatin powder, as compared to combining the powder with saline.

It is to be appreciated that embodiments of the methods and devices discussed herein are not limited in application to the details of construction and the arrangement of components set forth in this description or illustrated in the accompanying drawings. The methods and devices are capable of implementation in other embodiments and of being practiced or of being carried out in various ways. Examples of specific implementations are provided herein for illustrative purposes only and are not intended to be limiting. Also, the phraseology and terminology used herein is for the purpose of description and should not be regarded as limiting. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present devices and methods or their components to any one positional or spatial orientation.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description is by way of example only.

What is claimed is:

1. A kit for hemostasis, comprising:
a solution comprising a self-assembling peptide comprising between about 7 amino acids and 32 amino acids in an effective amount and in an effective concentration for use in forming a hydrogel under physiological conditions to promote hemostasis; and
a hemostatic powder miscible in the solution to form a mixture capable of promoting hemostasis on a wound having an initial bleeding score of 2 or higher, as assessed on the World Health Organization (WHO) Bleeding Scale.

2. The kit of claim 1, wherein the mixture is capable of promoting hemostasis on a wound having an initial bleeding score of 3 or higher, as assessed on the World Health Organization (WHO) Bleeding Scale.

3. The kit of claim 1, wherein the self-assembling peptide is selected from the group consisting of RADA16 and IEIK13.

4. The kit of claim 1, wherein the self-assembling peptide comprises KLD12.

5. The kit of claim 1, wherein the hemostatic powder comprise microspheres and/or micro-fibrils.

6. The kit of claim 1, wherein the hemostatic powder comprises a bio-absorbable material.

7. The kit of claim 6, wherein the hemostatic powder comprises collagen, gelatin, chitosan, polysaccharide, starch, hyaluronic acid, silk fibroin, or oxidized regenerated cellulose.

8. The kit of claim 1, wherein the hemostatic powder comprises a synthetic biomaterial.

9. The kit of claim 8, wherein the synthetic biomaterial is selected from the group consisting of: Poly(lactide-co-glycolide) (PLGA), (PLGA)-poly(ethylene glycol)-block-copolymer, and (PLGA-b-PEG).

10. The kit of claim 1, further comprising a syringe system for mixing the solution and the hemostatic powder.

11. The kit of claim 1, further comprising instructions for administering the solution and the hemostatic powder to a target site.

12. The kit of claim 1, wherein the instructions provide direction to mix the solution and the hemostatic powder in a ratio of about 0.1 to 20 mL solution per 1 g hemostatic powder by weight.

13. The kit of claim 12, wherein the instructions provide direction to mix the solution and the hemostatic powder in a ratio of about 0.5 to 7 mL solution per 1 g hemostatic powder by weight.

14. The kit of claim 11, wherein the instructions provide direction to apply a mixture of the solution and the hemostatic powder to the target site in excess, and then to cover the target site with gauze.

15. The kit of claim 14, wherein the instructions provide further direction to apply tactile pressure to the gauze.

16. The kit of claim 1, further comprising at least one of: a male luer-lock syringe, a female luer-lock syringe, a delivery nozzle, a bottle, a spreader, a container, and gauze.

17. The kit of claim 16, wherein an inner diameter of the delivery nozzle is about 0.5 mm to about 10 mm, and a length of the nozzle is from about 0.5 cm to about 30 cm.

18. The kit of claim 17, wherein the nozzle is flexible.

19. A macroscopic scaffold consisting essentially of a hemostatic powder and a plurality of self-assembling peptides, each of the self-assembling peptides comprising between about 7 amino acids and about 32 amino acids in an effective amount to promote hemostasis at a target area.

20. The macroscopic scaffold of claim 19, wherein the self-assembling peptide is selected from the group consisting of RADA16 and IEIK13.
